(19) United States
(12) Patent Application Publication
(10) Pub. No.: US 2010/0221812 A1
(43) Pub. Date: Sep. 2, 2010

(54) PRODUCTION OF GENETICALLY MODIFIED ACTINOMYCETES BY RECOMBINATION

(75) Inventors: Andreas Bechthold, Schallstadt (DE); Andriy Luzhetskyy, Freiburg (DE); Lutz Petzke, Freiburg (DE); Elisabeth Welle, Neuenburg (DE); Marta Fedoryshyn, Freiburg (DE); Martina Daum, Freiburg (DE); Simone Herrmann, Freiburg (DE)

Correspondence Address: SMITH PATENT CONSULTING, LLC, 515 East Braddock Road, Suite B, ALEXANDRIA, VA, 22314, US

(73) Assignee: ALBERT-LUDWIGS-UNIVERSITÄT FREIBURG, FREIBURG (DE)

(21) Appl. No.: 12/674,888

(22) PCT Filed: Feb. 12, 2008
(86) PCT No.: PCT/EP08/01055

§ 371 Date: Feb. 23, 2010

(30) Foreign Application Priority Data
Aug. 30, 2007 (EP) 07017025.3

(51) Int. Cl. C12N 1/21; C12N 15/74

(52) U.S. Cl. 435/252.3; 435/320.1

(57) ABSTRACT

The present invention relates to the recombinant modification of microorganisms, in particular actinomycetes. This is effected with the use of the recombination systems Dre-rox and Cre-lox. The present invention provides novel nucleotide sequences for the recombinases of the abovementioned recombination systems. Here, synthetic genes which have the codons preferred by actinomycetes and can therefore be used in these host organisms were produced.

With the method according to the invention for modifying microorganisms, it is possible to produce overproduction strains which are of economic importance for the production of medical active substances.

The present invention relates to nucleic acid molecules which can be replicated in host cells and contain novel DNA sequences which code for Dre or Cre recombinase. Furthermore, the invention relates to a recombination system for modifying DNA fragments (excision, integration, inversion or translocation) of the genome (target DNA) of actinomycetes. In addition, the invention relates to a method for producing modified actinomycetes, and the modified microorganisms themselves which were produced by this method.

In the present invention, actinomycetes are understood as meaning those gram-positive bacteria which have a high G+C ratio. In particular, they are those bacteria which are to be encountered naturally in the soil and possibly belong to the Streptomyces species.

Almost half of all drugs on the market are natural substances or are derived therefrom. A multiplicity of active substances is produced using microorganisms. Some successful examples on the market are the antibiotics erythromycin and tetracycline and the immunosuppressives cyclosporin, FK506 and rapamycin.

A method for producing natural substances or secondary metabolites is “combinatory biosynthesis”. In this method, artificial biosynthesis paths are created from a large, constantly growing collection of genes which are involved in syntheses of natural substances. First, the often labour-intensive and time-consuming construction of mutants of a bacterial strain is necessary for this purpose. These microorganisms should have an overproduction of secondary metabolites and therefore permit a cost-efficient method for producing active substances.

The genetic modification of microorganisms can be effected in a known manner by recombination. The recombinases of the Cre-lox (or Cre-loxP) and Dre-rox systems can catalyse the excision or integration and the inversion or translocation of DNA fragments which are present between specific identification sequences. Here, the recombinases Cre and Dre require no additional cofactors (Bucholz et al., Nucleic Acids Res. 1996, 24, 3118-3119; Huang et al., J. Bacteriol. 1997, 179, 6076-6083; Shimshek et al., Genesis, 2002, 32, 19-26).

In a recombination method of this type, it is necessary to introduce into the host cell and to express the nucleic acids which code for the recombinases. This can be effected, for example, by means of a plasmid, it being possible to control the expression of the recombinases with the use of inducible promoters. The recombinases can then bring about the modification of the genome of the host cell. The type of recombination is determined here by the arrangement of the identification sequences loxP and rox relative to one another on a DNA segment.

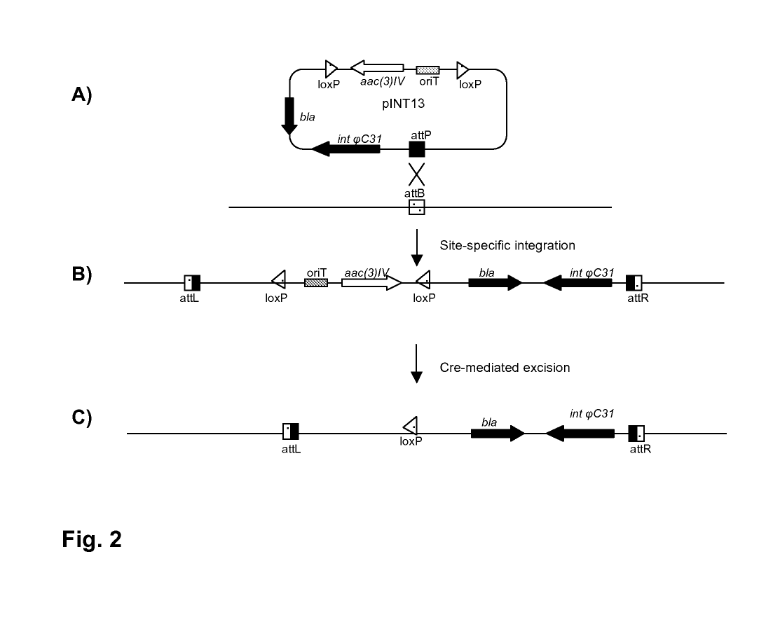

The introduction of identification sequences into the genome of the host cell can be effected by customary methods, for example by homologous recombination. In the presence of two identical identification sequences, the recombinase can bring about the excision of the DNA fragment which is present between these identification sequences, it then being possible to cyclize the excised DNA fragments. Furthermore, it is possible to introduce cyclic nucleic acid molecules which have an identification sequence, for example in the form of a plasmid, into the genome of the host cell. For this purpose, an identification sequence is also necessary in the genome host cell, whereupon the recombinase brings about an exchange of the nucleic acid strands at the identification sequences. The introduction of a cyclic nucleic acid molecule into E. coli strains is effected in a similar manner with the use of identification sequences attP (identification sequence of a bacteriophage) and attB (bacterial identification sequence, cf. FIG. 2). This process is catalyzed by two enzymes: the phage protein integrase and the bacterial Integration Host Factor (IHF). The phage genome is present after the integration as a prophage in the bacterial genome and is flanked by the newly formed recombination sites.

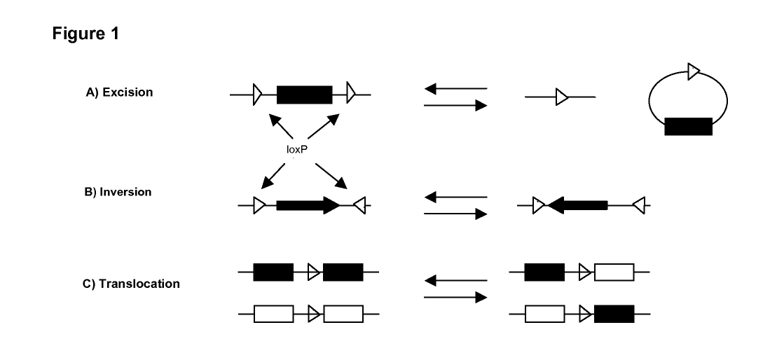

FIG. 1 shows the excision of a gene fragment (A), the inversion of a gene fragment (B) and the translocation of a gene fragment (C). Here, direction-dependent loxP identification sequences and Cre recombinase are used. The loxP identification sequences consist of a base region (eight base pairs) which is flanked by two inverted repeats (13 base pairs) which serve as identification sequences for the DNA binding of Cre (Mack et al., Nucleic Acids Res., 1992, 20, 4451-4455; Hoess et al., J. Mol. Biol., 1990, 216, 873-882). The recombination event is dependent only on these two constituents and is carried out with absolute reliability.

Of particular importance for the recombination by means of recombinases is the ability of the host cell to express the recombinases. The Dre- and Cre-mediated recombination is possible both in E. coli and in eukaryotic cells, including Drosophila, mouse, maize, rice, Arabidopsis and tobacco cells (Campo et al., Appl. Environ. Microbiol., 2002, 68, 2359-2367; Golic et al., Cell, 1989, 59, 499-509; Posfai et al., Nucleic Acids Res., 1994, 22, 2392-2398; Sauer and McDermott, Nucleic Acids Res. 2004, 18, 6086-6095).

Sauer et al., Nucleic Acids Research (2004), pages 6086-6095, describe the use of Cre recombinase of the bacteriophage P1 in various E. coli strains. Suzuki et al., Applied and Environmental Microbiology (2005), pages 8472-8480 and Appl. Microbiol. Biotechnol. (2005), pages 225-233, describe a Cre/loxP-mediated deletion system in Corynebacterium glutamicum. However, the system described there cannot be applied directly to other bacteria having a high G+C content.

The recombination systems described above can be introduced by means of plasmids into actinomycetes and replicated there. Here, the nucleotide sequences which code for the Cre and Dre recombinases can be cloned together with the necessary promoters and replication origins into a suitable plasmid. This plasmid can, for example, be introduced by means of conjugation into a microorganism and replicated there. Whether the recombinases are expressed in the host organisms is critical.

The recombination systems introduced into the host organism should be capable, for example, of introducing DNA fragments into the genome of a host organism or excising them from said genomes. In this way, it is possible, for example, to switch genes of the host organism on or off, this influencing the production of the desired active substances or secondary metabolites.

Particularly suitable microorganisms for the production of natural substances are the actinomycetes and in particular strains which belong to the Streptomyces genus. Recently, a method for using the Cre-lox system in Streptomyces coelicolor was developed (Khodakaramian et al., Nucleic Acids Res., 2006, 34, e20). However, this system can be used only to a limited extent in other strains. It is based on the phage φC31, which cannot transfix all strains. In addition, it is not simple to handle phages in association with Streptomycetes, so that routine use is not possible. Experiments to express the Flp recombinases in actinomycetes have not been successful to date (Khodakaramian et al., Nucleic Acids Res., 2006, 34, e20).

Furthermore, the heterologous expression of proteins is often difficult. It is observed that transcription and translation take place successfully but the folding of the proteins is unsuccessful. Proteases can recognize and destroy the foreign proteins. Surprisingly, it has now been possible to adapt the recombination systems described above to actinomycetes by optimizing the codon usage of the genes which code for the recombinases. By using codon usage of optimized synthetic genes, it is now possible to permit expression of the genes in actinomycetes which are essential for the recombination system. Consequently, the advantageous recombination methods can be used for actinomycetes.

Furthermore, it is not only disadvantageous if a system for recombination can be used only in a specific strain but it is also disadvantageous if only one system for recombination functions in this strain. Since it is not possible to incorporate an arbitrary number of identification sequences into a chromosome—this leads to undesired secondary effects—complex modifications which require the exchange of a plurality of gene fragments are not possible with a recombination system alone. It would therefore be desirable to permit complex modifications on microorganisms by enabling the use of different recombination methods in combination.

An object of the present invention is therefore to make it possible to use the Cre-lox or Dre-rox recombination systems for the actinomycetes genus. In particular, a combination of different recombination systems for producing an overproduction strain should be possible here.

This object is therefore achieved by the recombination systems according to the invention comprising novel nucleic acid sequences which code for the recombinases. The present invention therefore relates to nucleic acid molecules and associated identification sequences which can be replicated in host cells.

In a preferred embodiment, the nucleic acid molecule which can be replicated in host cells is characterized in that it contains at least one nucleic acid sequence selected from the group consisting of:
i) DNA sequences which have at least 80% identity with the nucleotide sequence SEQ ID No. 4 over the entire length and code for a Dre recombinase,
ii) DNA sequences which have at least 80% identity with the nucleotide sequence SEQ ID No. 5 over the entire length and code for a Cre recombinase,

In a preferred embodiment, the DNA sequences have at least 90% identity and more preferably at least 95% identity with the nucleotide sequences SEQ ID No. 4 and 5, respectively.

The nucleic acid molecule from the group i)-ii) which can be replicated in host cells has nucleotide triplets which represent those codons which are preferred by actinomycetes to an extent of at least 70%, preferably at least 80% and particularly preferably at least 90%.

The nucleic acid molecule which can be replicated in host cells can thus be preferably replicated in host cells such as an Escherichia coli strain or one or more Actinomycetes strains. Advantageously, it can be not only replicated but also expressed in an actinomycetes strain.

In a more preferred embodiment, the recombination system can be replicated in host cells, the host cells representing a Streptomycetes strain, Streptomyces lividans, Streptomyces coelicolor, Streptomyces albus, Streptomyces cinnamonensis, Actinoplanes sp. SE50/110, Streptomyces galilaeus, Streptomyces nodosus, Actinosynnema pretiosum subsp. Auranticum, Streptomyces virido-chromogenes Tü57, Streptomyces echinatus, Streptomyces avermitilis MA-4680, Micromonospora echinospora, Streptomyces sp. FR-008, Streptomyces chartreusis, Streptomyces bikiniensis, Streptomyces roseochromogenes subsp. oscitans DS12.976, Streptomyces griseus subsp. griseus, Streptomyces neyagawaensis, Streptomyces olindensis, Streptomyces rishiriensis DSM 40489, Streptomyces venezuelae, Streptomyces peucetius, Streptomyces olivaceus Tü2353, Saccharopolyspora erythraea, Micromonospora carbonacea var. Africana ATCC39149, Streptomyces griseoflavus Gö3592, Streptomyces ambofaciens, Streptomyces violaceoruber Tü22, Amycolatopsis orientalis, Amycolatopsis orientalis, Amycolatopsis mediterranei (balhimycina) DSM5908, Micromonospora echinospora ATCC15835, Streptomyces griseoruber, Streptomyces aureofaciaens Tü11, Streptomyces antibioticus Tü99, Streptomyces antibioticus Tü1718, Streptomyces sp. Tü2187, Streptomyces sp. Tü1156, Streptomyces sp. FU107, Streptomyces sp. FU36, Streptomyces venezuelae ISP5230, Streptomyces kanamyceticus, Streptomyces cyanogenus S136, Streptomyces aureofaciens Tü117, Streptomyces rochei, Streptomyces globisporus 1912, Streptomyces sp. AM-7161, Micromonospora megalomicea, Streptomyces argillaceus, Streptomyces carzinostaticus ATCC 15944, Streptomyces fradiae NCIB 8233, Streptomyces spheroides, NCIMB 11891, Streptomyces noursei ATCC11455, Streptomyces antibioticus, Actinoplanes teichomyceticus, Streptomyces natalensis, Streptomyces sp. Tü 6071, Streptomyces diastato-chromogenes Tü6028, Lechevalieria aerocolonigenes ATCC39243, Saccharothrix espanaensis, Micromonospora sp. Tü 6368, Streptomyces antibioticus Tü 6040, Streptomyces nogalater, Sorangium cellulosum So ce12, Saccharopolyspora, Spinosa, Streptomyces sp. TP-A0274, Streptomyces steffisburgensis NRRL 3193, Streptomyces griseus, Streptomyces fradice, Streptomyces fradiae Tü2717, and Streptomyces halstedii HC-34 being preferred.

In a further embodiment, the nucleic acid molecule which can be replicated in host cells is characterized in that it represents a plasmid.

In another embodiment, the nucleic acid molecule which can be replicated in host cells is characterized in that the plasmid has an ErmE or tipA promoter.

More preferably, the nucleic acid molecule which can be replicated in host cells is characterized in that the plasmid is selected from the group consisting of: pUWL-T-Dre, pUWL-A-Dre, pUWL-H-Dre, pNL1-Der, pML1-Dre and pAL1-Dre, pUWL-T-Cre, pUWL-A-Cre, pUWL-H-Cre, pNL1-Cre, pML1-Cre and pAL1-Cre.

The invention relates to a recombination system for the excision, integration, inversion or translocation of DNA fragments from/into the genome (target DNA) of actinomycetes, which recombination system has at least one nucleic acid molecule according to the invention and at least one identification sequence in the target DNA. More preferably, the recombination system comprises a DNA fragment which is to be inserted into the genome of the actinomycetes. More preferably, the DNA fragment to be inserted is introduced by means of a plasmid into the host cell. Preferably, the plasmid here comprises an identification sequence which corresponds to an identification sequence in the target DNA. More preferably, the plasmid additionally comprises the necessary replication origins for actinomycetes and for E. coli. Even more preferably, the DNA fragment to be inserted has a length of at least 1000 base pairs.

The recombination system has an identification sequence selected from the group consisting of: loxP and rox identification sequences.

In a more preferred embodiment, the recombination system is characterized in that the target DNA contains at least two identification sequences selected from the group consisting of loxP and rox identification sequences. More preferably, the target DNA contains two different identification sequences from the abovementioned group.

In a more preferred embodiment, the recombination system is characterized in that the nucleic acid molecules which code for the recombinases are present in a plasmid and the plasmid is replicated in an Escherichia coli strain and/or introduced into an Actinomycetes strain and is replicated and/or expressed there.

Furthermore, the invention relates to a method for producing modified actinomycetes, comprising the following steps:
a) insertion of at least one identification sequence into the genome (target DNA) of the actinomycetes, the identification sequences being selected from the group consisting of loxP and rox identification sequences,
b) introduction of at least one plasmid into the actinomycetes, the plasmid representing a replicatable nucleic acid molecule containing a codon-optimized recombinase, and
c) replication of the plasmid from b) and expression of the recombinase which is specific for the identification sequence(s) of the target DNA, excises, translocates or inverts a DNA fragment of the target DNA or inserts a DNA fragment into the target DNA.

In another embodiment, the method is characterized in that at least two identification sequences selected from the group consisting of loxP and rox identification sequences are inserted into the target DNA.

More preferably, the method is characterized in that at least two different nucleic acid molecules according to the invention are used. Here, it is preferable if at least two different DNA fragments can be inserted by at least two different recombination systems according to the invention into the target DNA or excised from said target DNA.

A more preferred embodiment is the use of a recombination system for the excision, integration, inversion or translocation of DNA fragments in actinomycetes.

Furthermore, the invention relates to a modified microorganism produced by the method according to the invention.

The nucleic acid molecules according to the invention firstly permit the production of plasmids which can be replicated in actinomycetes and streptomycetes. The expression of the novel nucleotide sequences or nucleic acid molecules according to the invention in the host organisms leads to the recombinases which permit a modification of the genome of the host cell by recombination.

It is particularly advantageous that the recombination systems Cre-lox and Dre-rox are now suitable for all actinomycetes. As a result, it is possible in particular to combine the different systems for recombination with one another in order to extend the possibilities with regard to the exchange of a plurality of fragments in a genome.

The method according to the invention furthermore advantageously permits the deletion and the simultaneous cloning of biosynthesis gene clusters. This in turn permits the rapid generation of mutants and hence the rapid and efficient generation of novel natural substances.

The method according to the invention makes it possible for overproduction strains which produce natural substances, active substances or secondary metabolites to be produced in a very short time.

Below, the recombination systems with the nucleotide sequences or nucleic acid molecules according to the invention are explained in detail.

“Target DNA” as used here designates the genome of the actinomycetes or an extrachromosomal plasmid of the actinomycetes which is to be modified. In the method according to the invention, at least one identification sequence is inserted into the target DNA in a first step. These identification sequences can be recognized by the recombinases, whereupon the recombinases bring about the modification of the target DNA. The insertion of loxP and rox identification sequences can be effected by techniques which are known to the person skilled in the art (cf. examples).

One possibility for introducing identification sequences into the genome of the host cell is shown in FIG. 2. Here, first a plasmid is introduced into the microorganism, in this case of the Streptomyces lividans strain. This plasmid (FIG. 2A) comprises the identification sequences (loxP) and a bacterial phage recombination site (AttP) which can bind to the attB (attachment site on the bacterial chromosome) of the chromosome of the host cell. Recombination takes place and the nucleic acid molecule of the plasmid is integrated into the genome of the host cell. As is evident from FIG. 2, two loxP identification sequences are now present in the target DNA. In FIG. 2, part A) is a schematic representation of the plasmid with the designation pINT13. In line B), the integration of the plasmid pINT13 into the chromosome of S. lividans is shown.

FIG. 2 C) shows the removal of an apramycin resistance gene (aac(3)IV), which is flanked by the two loxP sites, with the aid of the Cre recombinase. loxP is the identification sequence for the Cre recombinase. For the rest, the abbreviations in FIG. 2 denote: oriT: Origin of Plasmid transfer, bla: ampicillin resistance gene; intφC31: gene coding the integrase of the phage φC31, which is required for the integration of the plasmid pINT13 into the chromosome of S. lividans; attP: attachment site of the phage φC31; attB: φC31 attachment site in the S. lividans chromosome.

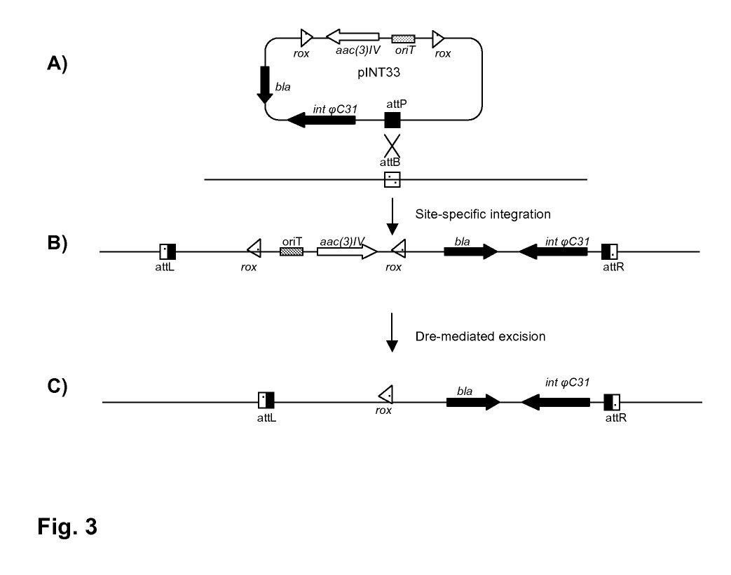

FIG. 3 illustrates the activity of the Dre recombinase in Streptomyces lividans. FIG. 3 A) shows a map of the plasmid pINT33, where rox represents the target sequence for the Dre recombinase. bla denotes the ampicillin resistance gene and aac(3)IV denotes the apramycin resistance gene. oriT is the origin of the plasmid transfer, int φC31 is the gene coding for the integrase of the phage φC31, which is required for the integration of the plasmid pINT33 into the chromosome of the target actinomycetes. attP is the attachment site of the phage φC31 and attB-φC31 is the attachment site in the actinomycetes chromosome. After a specific integration, the plasmid is integrated into the chromosome of the target organism S. lividans. This is shown schematically in FIG. 3 B). If the integrase Dre is correctly expressed in the target organism, excision of the nucleic acid sequence (in this case oriT and aac(3)IV) present between the two rox sites now takes place by recombination. The recombination product is shown in FIG. 3C.

Preferably, the target DNA contains, after insertion of identification sequences, at least one, more preferably at least two, even more preferably at least three and most preferably at least four identification sequences which can be recognized by a recombinase. More preferably here, the target DNA then has at least two identification sequences which in each case can be recognized by different recombinases. More preferably, at least three, more preferably at least four, even more preferably at least five identification sequences are inserted into the target DNA, at least two identification sequences being capable of being recognized by different recombinases.

Recombinases are required for carrying out modifications of the target DNA. These are provided by expression of the nucleic acid molecules according to the invention which code for these proteins. These nucleic acid molecules are synthetic genes which were produced by codon usage optimization. Here, the codons preferred by actinomycetes were used. The codons preferred by actinomycetes are shown in table 1.

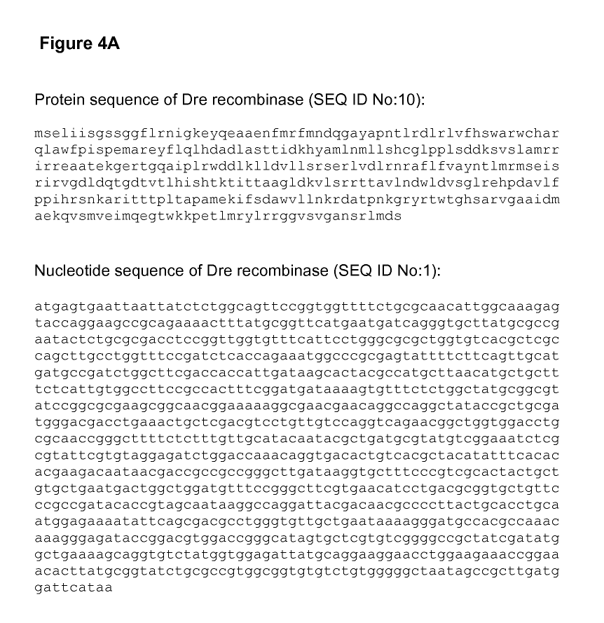

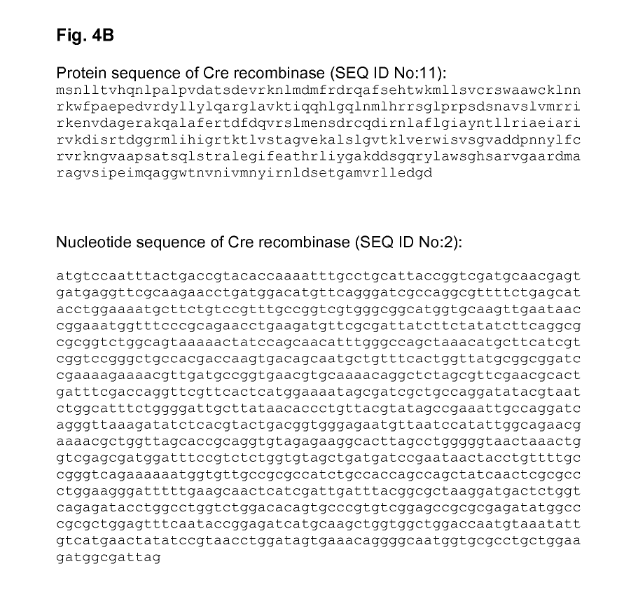

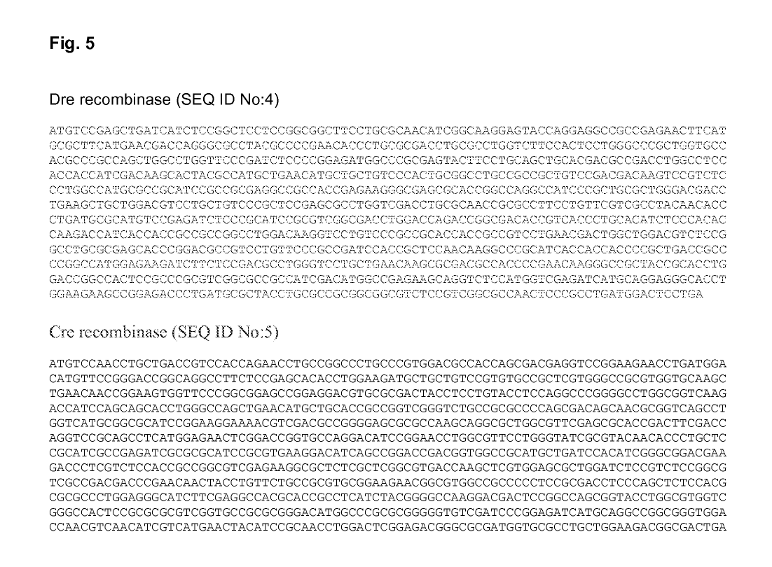

In order to obtain the synthetic genes, the triplets in the natural genes of the Cre and Dre recombinases which are not preferred by actinomycetes were exchanged for codons which are preferred by actinomycetes. The natural nucleotide sequences of the Dre recombinase (SEQ ID No: 1, gene bank No. AAV84949), of the Cre recombinase (SEQ ID No: 2, gene bank No. YP_{—}006472), are known. FIGS. 4A and 4B show the sequences of the recombinases and recombinase genes (SEQ ID No:10-12). By exchanging all those codons of the natural nucleotide sequences which are not preferred by actinomycetes for codons which are preferred according to table 1 by actinomycetes, the nucleotide sequences, according to the invention, of the Cre recombinase (SEQ ID No:4) and of the Dre recombinase (SEQ ID No:5) were obtained. FIG. 5 shows the nucleotide sequences according to the invention.

Methods for producing synthetic nucleic acid molecules according to the invention having the nucleotide sequences according to the invention are known to the person skilled in the art and are described in detail in the literature (Hoover and Lubkowski, Nucleic Acids Res., 2002 30 (10), e43). The software “DNAWorks” (http://molbio.info.nih.gov/dnaworks) was used for deriving the primary sequences. This program calculates the “optimal primer” starting from the desired amino acid sequence, the data from a type-specific codon usage table (table 1) and the desired length and melting point of the oligonucleotides.

In order to be able to carry out the method according to the invention for modifying actinomycetes, it is not necessary to adapt the codon usage 100% to the actinomycetes. Programs for creating DNA sequences which are optimized for codon usage are known to the person skilled in the art (for example “Prot2DNA”). The synthesis of the calculated sequences can be effected, for example, with the use of primers having 20 base pairs. The nucleotide sequences according to the invention therefore have an identity of at least 80%, more preferably of at least 85%, more preferably of at least 90%, even more preferably of at least 95%, more preferably of at least 98% and most preferably of 100% with the nucleotide sequences SEQ ID No:4-5.

The expression that a DNA sequence has an “identity with a nucleotide sequence” means that, with the use of a customary program for comparing nucleotide sequences, with standard parameters, the sequences to be compared have the specified percentage agreement.

Even more preferably, the nucleic acid molecules have, to an extent of at least 70%, those codons which are preferred by actinomycetes and are shown in table 1. More preferably, the nucleic acid molecules have at least 75%, more preferably at least 80%, even more preferably at least 85%, even more preferably at least 90% and most preferably at least 95% of codons preferred by actinomycetes and shown in table 1.

The transformation (take-up of DNA into prokaryotic cells) of the host cells takes place with the nucleic acid molecules according to the invention, the DNA fragments be inserted and the identification sequences, preferably with the use of plasmids. Here, it is firstly possible to use plasmids which contain the nucleotide sequences which code for the recombinases. Furthermore, plasmids can be used for inserting identification sequences or DNA fragments into the genome of the host cell.

These plasmids preferably carry genes which make it possible to introduce DNA fragments into actinomycetes: in the case of replicating vectors (for example: pUWL-T): on-Rep (E. coli), replication origin: on-Rep (Actinomycet), replication origin; antibiotics resistance gene (E. coli); antibiotics resistance gene (Actinomycet); oriT—sequence necessary for conjugation of E. coli to the actinomycetes (this is not required in the case of transformations, use of pUWL201 then possible).

In the case of integrating vectors: (for example: pint): intφC31—integrase-coding gene of the phage φC31 necessary for the integration of the vector into the genome; attP, identification sequence of the phage C31 necessary for the integration of the vector into the genome; ori-Rep (E. coli), replication origin; antibiotics resistance gene (E. coli); antibiotics resistance gene (Actinomycet); oriT—sequence necessary for the conjugation of E. coli to the actinomycetes (Bierman et al., Gene 1992, 116, 43).

This occurs with the so-called conjugation in which a plasmid is introduced by an E. coli strain into streptomycetes. Here, the nucleotide sequences according to the invention are additionally preferably present behind an ErmE promoter, which represents a constitutive promoter. As a result, the gene is continuously expressed. It is additionally preferred if the plasmid comprises a replication origin for actinomycetes. It is additionally preferred if it also contains a replication origin for the replication in E. coli. It is additionally preferred if the plasmid also contains an antibiotic resistance gene, such as, for example, the thiostrepton resistance gene (tsr), the apramycin resistance gene (apr), the hygromycin resistance gene (hyg). It is additionally preferred if the plasmid also contains an inducible promoter (for example a promoter inducible with thiostrepton) (Takano et al., Gene 1995, 166, 133.)

Suitable plasmids and necessary promoters, replication origins, etc are known to the person skilled in the art and can be selected therefrom for the transformation or conjugation of actinomycetes. Particularly preferred plasmids which can be used according to the invention are pUWL201, pSET152, pKC1218 etc. (Bierman et al., Gene 1992, 116, 43.)

Plasmids preferred according to the invention are pUWL-T, pUWL-A, pUWL-H, pNL1, pML1 and pAL1. However, a plasmid quite possibly contains one, two or three genes which code for different recombinases. With the recombination systems according to the invention, the excision, integration, inversion or translocation of DNA fragments is possible.

The integration of the identification sequences is carried out with plasmids which contain no ori-Rep (replication origin actinomycetes), no intφC31 gene and no attP sequence. Genes which code for recombinases are introduced into the strains with replicatable plasmids which contain no intφC31 gene and no attP sequence, or they are introduced into the strains with integrative vectors, which contain an intφC31 gene and an attP sequence, and an inducible promoter. There, the vectors can be integrated into the chromosome of the target organism.

The term “excision” of DNA fragments means that the nucleotide sequence between two identification sequences of the target DNA is excised by the recombinases.

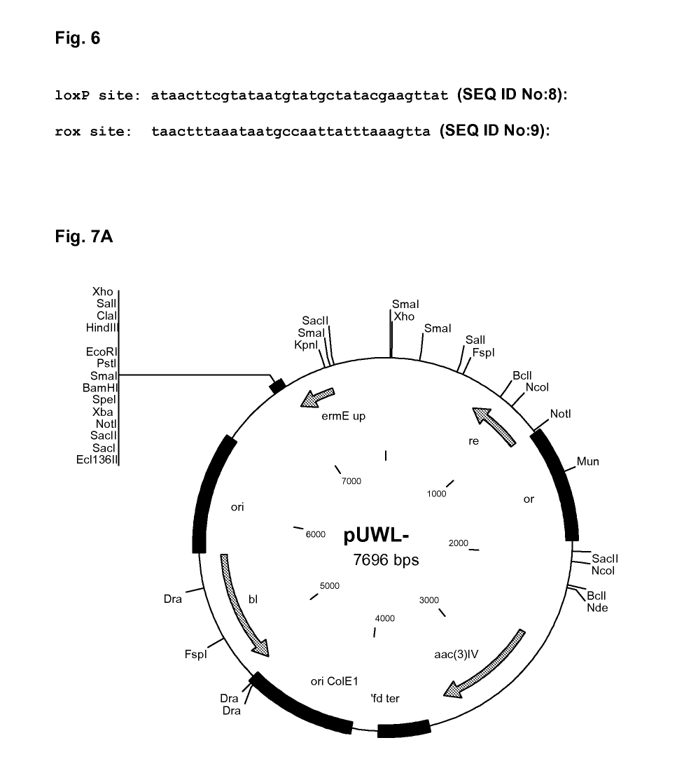

The identification sequence for the Cre recombinase is the loxP identification sequence with SEQ ID No:7, and the identification sequence of the Dre recombinase is the rox identification sequence with SEQ ID No:8. The synthesis of the identification sequences or of the corresponding nucleic acid molecules can be effected by customary methods and is known to the person skilled in the art (Hunkapiller et al., Nature 1984, 310, 105.). The loxP- and rox sequences are shown in FIG. 6.

The expression “integration” means that the DNA fragment to be inserted is adjacent to an identification sequence in a nucleic acid molecule and is integrated by the recombinase into the target DNA, the target DNA likewise having this identification sequence.

The expression “inversion” means that the DNA fragment to be inverted is adjacent to an identification sequence in a nucleic acid molecule and is inverted by the recombinase, i.e. turned around. Thus, the orientation of the DNA fragment changes.

The expression “translocation” means that a DNA fragment on the target DNA adjacent to an identification sequence exchanges position with a further DNA fragment on the same target DNA, likewise adjacent to an identification sequence.

The expression “DNA fragments to be inserted”, as used here, comprises any DNA sequences independently of their number of base pairs. Analogously, any nucleic acid molecule or any DNA fragment between two identification sequences can be excised from the target DNA. The DNA fragment to be inserted can preferably be introduced by means of a plasmid into the host cell. Here, the plasmid additionally preferably has an identification sequence which corresponds to an identification sequence in the target DNA. Furthermore, the plasmid additionally preferably contains a replication origin for actinomycetes and more preferably a replication origin for E. coli.

The expression “host cell” and “host organism” designates cells which are capable of expressing the nucleic acid molecules according to the invention. These are all microorganisms in which the preferred codons are the same as those in the case of the actinomycetes (cf. table 1). In particular, the expression “host cells” or “host organism” therefore preferably comprises actinomycetes, more preferably streptomycetes (as a genus of actinomycetes), more preferably the specific strains Streptomyces lividans, Streptomyces coelicolor, Streptomyces albus, Streptomyces cinnamonensis, Actinoplanes sp. SE50/110, Streptomyces galilaeus, Streptomyces nodosus, Actinosynnema pretiosum subsp. Auranticum, Streptomyces virido-chromogenes Tü57, Streptomyces echinatus, Streptomyces avermitilis MA-4680, Micromonospora echinospora, Streptomyces sp. FR-008, Streptomyces chartreusis, Streptomyces bikiniensis, Streptomyces roseochromogenes subsp. oscitans DS12.976, Streptomyces griseus subsp. griseus, Streptomyces neyagawaensis, Streptomyces olindensis, Streptomyces rishiriensis DSM 40489, Streptomyces venezuelae, Streptomyces peucetius, Streptomyces olivaceus Tü2353, Saccharopolyspora erythraea, Micromonospora carbonacea var. Africana ATCC39149, Streptomyces griseoflavus Gö3592, Streptomyces ambofaciens, Streptomyces violaceoruber Tü22, Amycolatopsis orientalis, Amycolatopsis orientalis, Amycolatopsis mediterranei (balhimycina) DSM 5908, Micromonospora echinospora ATCC15835, Streptomyces griseoruber, Streptomyces aureofaciaens Tü11, Streptomyces antibioticus Tü99, Streptomyces antibioticus Tü1718, Streptomyces sp. Tü2187, Streptomyces sp. Tü1156, Streptomyces sp. FU107, Streptomyces sp. FU36, Streptomyces venezuelae ISP5230, Streptomyces kanamyceticus, Streptomyces cyanogenus S136, Streptomyces aureofaciens Tü117, Streptomyces rochei, Streptomyces globisporus 1912, Streptomyces sp. AM-7161, Micromonospora megalomicea, Streptomyces argillaceus, Streptomyces carzinostaticus ATCC 15944, Streptomyces fradiae NCIB 8233, Streptomyces spheroides, NCIMB 11891, Streptomyces noursei ATCC11455, Streptomyces antibioticus, Actinoplanes teichomyceticus, Streptomyces natalensis, Streptomyces sp. Tü 6071, Streptomyces diastato-chromogenes Tü6028, Lechevalieria aerocolonigenes ATCC39243, Saccharothrix espanaensis, Micromonospora sp. Tü 6368, Streptomyces antibioticus Tü6040, Streptomyces nogalater, Sorangium cellulosum So ce12, Saccharopolyspora, Spinosa, Streptomyces sp. TP-A0274, Streptomyces steffisburgensis NRRL 3193, Streptomyces griseus, Streptomyces fradice, Streptomyces fradiae Tü2717, and Streptomyces halstedii HC-34.

The method according to the invention for producing modified microorganisms comprises the insertion of at least one identification sequence, preferably of two identification sequences, into the genome (target DNA) of the actinomycetes (step (a)), the introduction of at least one plasmid with the nucleic acid molecule according to the invention into the actinomycetes (step (b)), and the replication of the plasmid and expression of the recombinases which modifies/modify the target DNA (step (c)).

Step (a): In this step, identification sequences are inserted into the target DNA, the identification sequences being selected from the group consisting of loxP and rox identification sequences. The insertion of the identification sequences is preferably effected with the use of plasmids. The insertion of the nucleic acid molecules which contain the identification sequences can be effected, for example, via homologous recombination with the use of homologous DNA, which is preferably likewise present in the plasmid. Also preferably, the plasmid furthermore contains necessary promoters and replication origins for E. coli strains.

Step (b): In a subsequent step, the nucleic acid molecules according to the invention are introduced into the actinomycetes. Preferably, this is likewise effected with the use of plasmids. The introduction of plasmids into the actinomycetes is effected as in step (a) by means of transformation (or conjugation). Preferably, these plasmids comprise the necessary promoters and replication origins for actinomycetes and E. coli strains, more preferably for actinomycetes.

Step (c): In a subsequent step, the plasmid from step (b) is replicated in the actinomycetes. The replication of the plasmids in the steps (a) and (b) can be achieved with the use of customary conditions. Furthermore, the plasmids are expressed in the actinomycetes and the recombinases thus produced. The recombinases then recognize the identification sequences in the target DNA and bring about localized changes of the target DNA.

If at least one DNA fragment is inserted into the target DNA in step (c) of the above method, a further plasmid which contains the DNA fragment to be inserted and an identification sequence is additionally preferably used for this purpose, the identification sequence corresponding to the target DNA. If more than one DNA fragment is inserted into the target DNA, a plurality of plasmids can be used for this purpose. Preferably, these plasmids additionally contain the necessary promoters and replication origins for the replication in actinomycetes.

The conditions for transformation (or conjugation) of host cells are known to the person skilled in the art from the literature (Kieser et al., Practical Streptomyces Genetics, The John Innes Foundation, Norwich, 2000).

For the excision of DNA fragments, the presence of at least two identical identification sequences for a specific recombinase is required.

Preferably, different recombination systems or recombinases are used for the insertion of more than one DNA fragment. Here, preferably at least two different identification sequences, selected from the loxP and rox recombination identification sequences, are inserted into the target DNA. This permits, for example, the subsequent insertion of two different DNA fragments which are adjacent to different identification sequences, it being possible, for example, to insert one DNA fragment with the use of the rox identification sequence and of the Dre recombinase and the other DNA fragment, for example, with the use of the loxP identification sequence and the Cre recombinase. The method for modifying microorganisms is described in more detail in the examples.

In addition, it is preferable if a marker gene for identification and selection of the modified host cells is likewise introduced into the target DNA. Such marker genes are known to the person skilled in the art and are described in the literature (DeWet et al., Mol. Cell. Biol., 1987, 7, 725-737; Goff et al., EMBO J., 1990, 9, 2517-2522; Kain et al., BioTechniques, 1995, 19, 650-655; Chiu et al., Current Biology, 1996, 6, 325-330). Suitable marker genes are, for example, genes which mediate antibiotic resistance, such as, for example, apramycin, thiostrepton, chloramphenicol, methodrexate, hygromycin, streptomycin, spectinomycin and bleomycin (Herrera Estrella et al., EMBO, J. 1983, 2, 987-992; Herrera Estrella et al., Nature, 1983, 303, 209-213; Meijer et al., Plant. Mol. Biol., 1991, 16, 807-820; Waldron et al., Plant Mol. Biol., 1985, 5, 103-108; Zhijian et al., Plant Science, 1995, 108, 219-227; Jones et al., Mol. Gen. Genet., 1987, 210, 86-91; Bretagne-Sagnard et al., Transgenic Res., 1996, 5. 131-137; Hille et al., Plant Mol. Biol., 1990, 7, 171-176; Guerineau et al., Plant Mol. Biol., 1990, 15, 127-136).

The recombination system, according to the invention, of the present invention for modifying microorganisms comprises at least one nucleic acid molecule according to the invention, which codes for a recombinase. Furthermore, it contains a target DNA which has at least one identification sequence. All nucleic acid molecules according to the invention, plasmids and any target DNA described above can be used for the recombination system. Preferably, apramycin or hygromycin are used as antibiotics.

The modified microorganism according to the invention can be produced with the use of the methods described here. The biosynthesis gene cluster (biosynthesis gene cluster I) for the natural substance is removed by the novel technology from a known overproduction strain which can produce a certain natural substance in large amounts. The total biosynthesis gene cluster (biosynthesis gene cluster II) of a natural substance whose synthesis in large amounts is desired is likewise cloned by the novel technology and then introduced into the modified overproduction strain. It may be possible to exchange promoters on the biosynthesis gene cluster II beforehand. Thus, the synthesis performance for the natural substance I is applied to the synthesis performance for the natural substance II.

FIG. 1 relates to Cre-lox reactions as a function of the orientation and spatial arrangement of the loxP interfaces. Paired loxP identification sequences (triangles) are direction-dependent and can be localized in cis (identical DNA strand) of a trans (different DNA strands) arrangement.
(A) If the loxP identification sequences flank a DNA segment in cis arrangement and are oriented in the same direction, Cre recombination mediates the excision and the circularization of the segment.
(B) If the loxP identification sequences flank a DNA segment in cis arrangement and are oriented in opposite directions, Cre recombination mediates an inversion of the segment.
(C) If the loxP identification sequences are localized on different DNA strands and oriented in the same direction, Cre recombination mediates a shift of the segment to the respective other strand.

FIG. 2 shows the activity of the Cre recombinase in Streptomyces lividans. It is shown schematically how intφC31, the gene coding for the integrase of the phage φC31, is introduced into the chromosome of S. lividans.

FIG. 3 shows the activity of the Dre recombinase in Streptomyces lividans. FIG. 3A shows a schematic diagram of the plasmid pINT33. FIG. 3B shows the integration of the plasmid pINT33 into the chromosome of S. lividans via attB, which represents the attachment site for the phage φC31 in the actinomycetes chromosome.

FIG. 3C shows the removal of the apramycin resistance gene which is flanked by the rox sites with the aid of the Dre recombinase. The rox sites are the target sequences for the Dre recombinase. The example shows the insertion of the ampicillin resistance gene (bla). The gene intφC31 is the gene coding for the integrase of the phage φC31, which is required for integration of the pINT33 plasmid into the chromosome of actinomycetes.

FIGS. 4A and 4B show the original sequences (gene sequences SEQ ID No:1 and 2 and the protein sequences SEQ ID No:10-11) of the recombinases Cre and Dre.

FIG. 5 shows the gene sequences according to the invention of the recombinases Dre (SEQ ID No:4) and Cre (SEQ ID No:5).

FIG. 6 shows the identification sequences loxP (SEQ ID No:8) and rox (SEQ ID No:9).

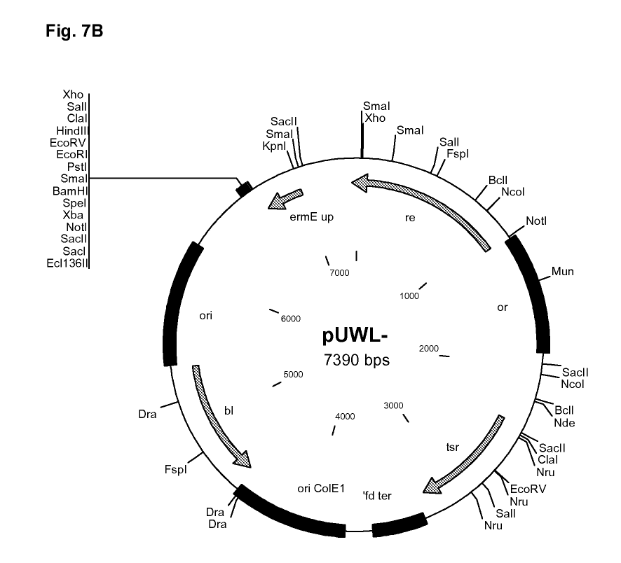

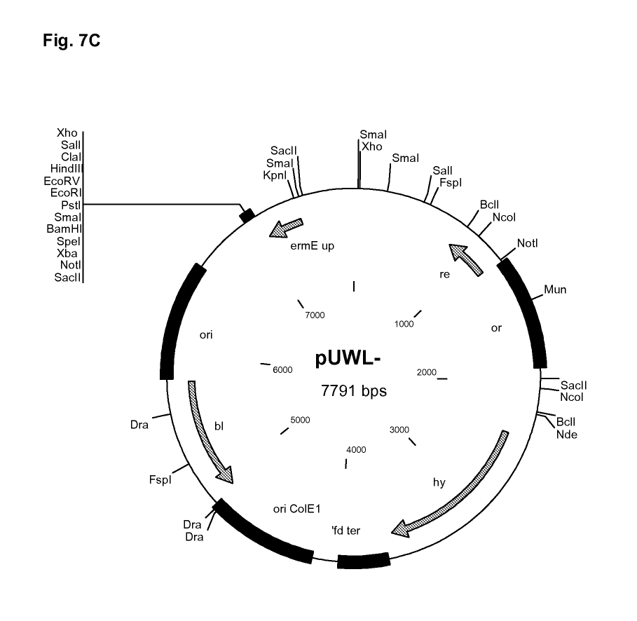

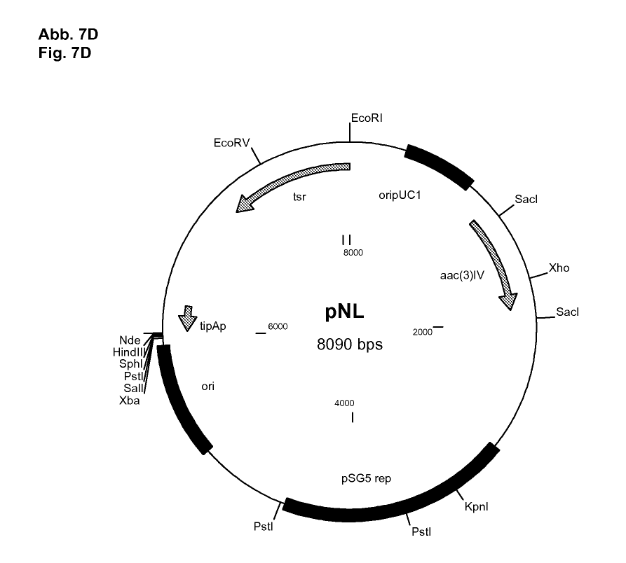

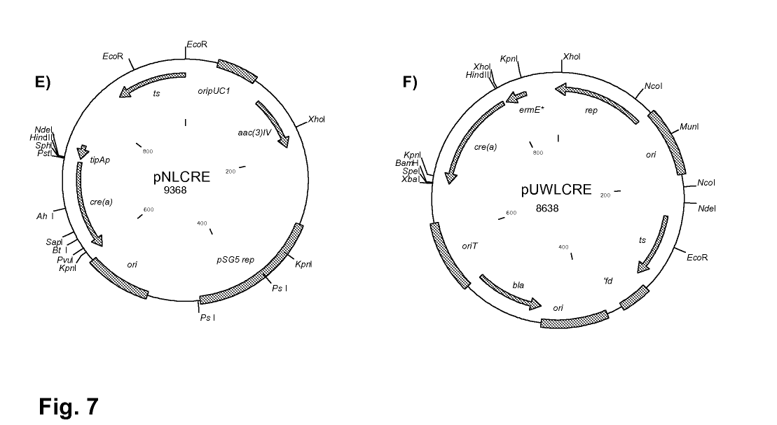

FIGS. 7A-D show the plasmids pUWL-T, pUWL-A, pUWL-H, and pNL1 according to the invention, which can be used for introducing and for expressing the recombinases in actinomycetes. pUWL-T was obtained from pUW201 by introduction of oriT (sequence important for the conjugation process). pUWL-A and pUWL-H formed from pUWL-T by exchange of the antibiotics resistance genes (T, thiostrepton; A, apramycin, H, hygromycin). pNL1 formed from pIJ8600 (cloning of an Eco/RI-BamHI fragment which contains the tipA promoter and the thiostrepton resistance gene) and pKC1139 (cloning of an Eco/RI-Bg/II fragment which contains the temperature-sensitive replicon pSG5 and the apramycin resistance gene aac(3)IV).

Abbreviations used:

ermE, strong promoter for the expression in actinomycetes; bla, β-lactamase; on ColE1, replication origin necessary for the replication in E. coli; on pUC18, replication origin necessary for the replication in E. coli; tsr, thiostrepton resistance gene; aac(3)IV, apramycin resistance gene, hyg, hygromycin resistance gene, rep^{PIJ101}, replication origin necessary for the replication in actinomycetes. pSG5 rep, temperature-sensitive replication origin necessary for the replication in streptomycetes, tipA, thiostrepton promoter.

FIGS. 7E and 7F show maps of plasmids which comprise the synthetic codon-optimized cre(a) gene according to the invention. FIG. 7E schematically shows the plasmid pNLCRE and FIG. 7F schematically shows the plasmid pUWLCRE.

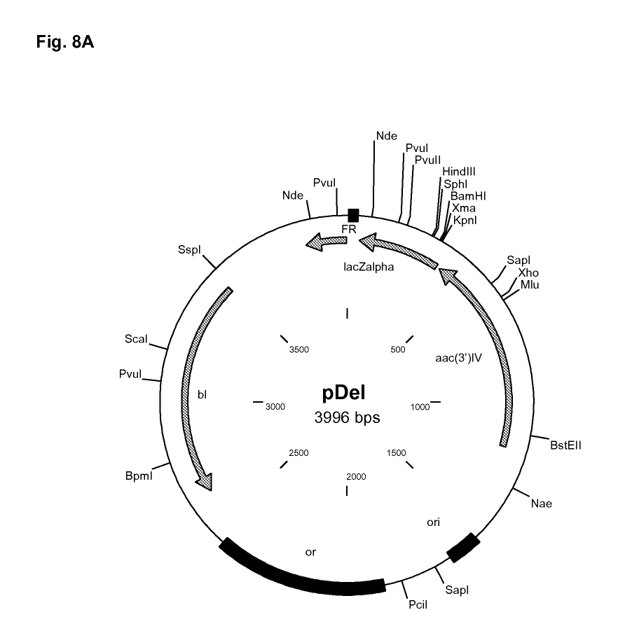

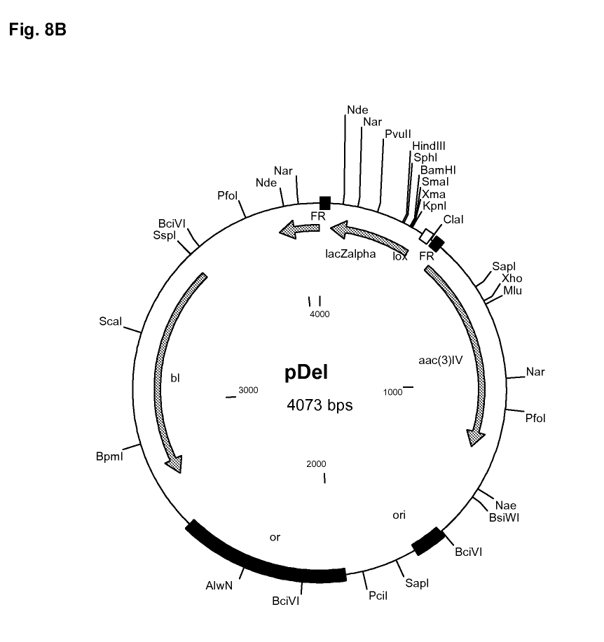

FIGS. 8A and B show the plasmids according to the invention which can be used for deleting and for cloning a natural substance biosynthesis gene cluster.

Plasmids pDel1 and pDel2 are produced by PCR fusion. Starting vectors for the PCR were pUC19 and pIJ773 (Khodakaramian et al., Nucleic Acids Res., 2006, 34, e20).

Abbreviations used:

FRT, identification sequence of the Flp recombinase; carb, β-lactamase gene; oriT, sequence necessary for the conjugation of E. coli to the actinomycetes; on, sequence necessary for the replication in E. coli; lacZ and lacZ′, sequences of the β-galactosidase gene; loxP, identification sequence of the Cre recombinase (additional DNA element which is currently not required); apra, apramycin resistance gene. FIG. 4B shows the generation of a mutant of the phenalinolactone producer with simultaneous cloning of the phenalinolactone biosynthesis gene cluster. The Frt identification sequences are shown as triangles. Am^{r}, apramycin resistance gene; ori^{pUC}, replication origin, necessary for the replication in E. coli.

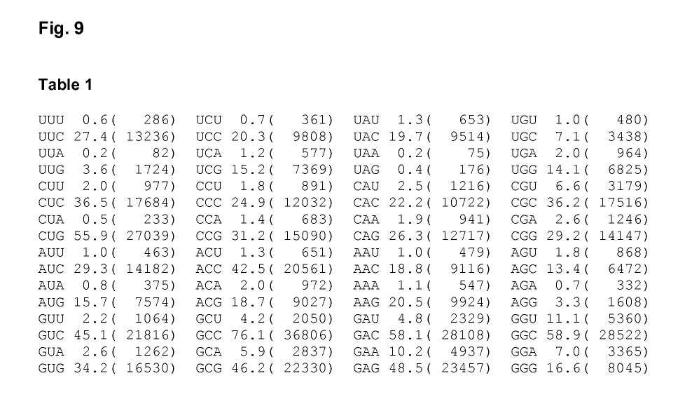

FIG. 9 shows table 1. Table 1 shows the codon usage of actinomycetes. (http://www.nih.go.jp/˜jun/act/codon/104.html).

### EXAMPLE 1

### Synthesis of the Nucleic Acid Molecules According to the Invention

The synthesis of the molecules was carried out by the company DNA 2.0 (Cre) and the company Genscript (Dre).

### EXAMPLE 2

### Synthesis of the Plasmids with the Nucleic Acid Molecules According to the Invention

The strain Streptomyces lividans TK24 was used as a model strain in order to carry out the expression of the native Cre and the synthetic Cre(a) genes. For cloning, the E. coli strain DH5a was used, and the strain ET12567/pUZ8002 was used for bringing about the conjugative transfer of non-methylated nucleic acid from E. coli to S. lividans.

With the aid of customary methods of molecular biology, the two plasmids according to the invention, pNLCRE and pUWLCRE, as shown in FIGS. 7E and F were produced. Both plasmids comprise the synthetic Cre(a) genes.

### EXAMPLE 3

### Insertion of Identification Sequences into the Target DNA

Plasmids can be introduced into the actinomycetes strains by means of conjugation or transformation. The experiments are described in Kieser et al. (Kieser et al., Practical Streptomyces Genetics, The John Innes Foundation, Norwich, 2000).

### EXAMPLE 4

### Induction of the Recombination

The induction of the recombinase which was cloned behind the tipA promoter is effected by addition of thiostrepton (3.5 μg/ml).

### EXAMPLE 5

### Insertion and Excision of an Apramycin Resistance Cassette

According to the invention, it was found that the native Cre gene comprises of 145 codons which are used, for example, in the case of Streptomyces coelicolor with a frequency of less than 1%. These codons can therefore be regarded as rare codons. Because the gene expression of rare codons can lead to errors in the translation, genes modified according to the invention were discarded and synthesized (cre(a)) using the preferred codons of Streptomyces coelicolor. The genes of the genus Streptomyces have a GC content which is typically greater than 70%. The genes according to the invention preferably have a G+C content of more than 65%. A G+C content of 67.7% for cre(a) is particularly preferred. In order to compare the activity and efficiency of the native recombinase cre and of the codon-optimized synthetic cre recombinase cre(a), plasmid pINT13, which comprises the integration system of the phage φC31 and has the apramycin resistance gene flanked by loxP sites was constructed (FIG. 2). The plasmid pINT13 was introduced by conjugation into S. lividans and integrated in a stable manner via the φC31-attB site. S. lividans with the plasmid pINT13 was cultured under non-selective conditions and 230 clones were checked for apramycin resistance. 100% of the clones comprised pINT13. In order to test the activity of the Cre recombinase, the plasmids pUWLCRE, pUWLCREN, pNLCRE and pNLCREN were transformed into S. lividans pINT13. After passage of S. lividans pINT13 containing pUWLCRE under non-selective conditions, 743 clones were tested for resistance to apramycin. Surprisingly, every clone tested has lost the resistance marker.

In contrast, only three sensitive clones were found among 198 clones tested when pUWLCREN was used instead of pUWLCRE.

The efficiency of the synthetic gene cre(a) after expression by the plasmid pNLCRE was also checked. All 227 clones of S. lividans pINT13 and the plasmid pNLECRE did not grow on plates containing apramycin. This indicates a loss of the apramycin resistance gene with a frequency of 100%.

Accordingly, only a single clone from 169 clones which contained the native Cre gene expressed by the plasmid pNLCREN did not grow on plates containing apramycin. This indicates a loss of the apramycin resistance gene with a frequency of less than 1%.

Since the apramycin resistance gene is laterally flanked by loxP, the loss of the resistance is the consequence of the Cre activity.

By sequencing the chromosomal segment of the DNA of S. lividans, it was determined that the gene was excised specifically on the roxP sides. This experiment clearly shows that the synthetic gene cre(a) used according to the invention can be efficiently expressed in S. lividans and performs the function, whereas this is not the case with the wild type gene (cre).

### EXAMPLE 6

### Production of an Overproduction Strain

For producing an overproduction strain, an actinomycetes strain which produces large amounts of a natural substance is used. The biosynthesis gene cluster is removed from this strain by the methods described. Biosynthesis gene clusters of the natural substances which are now to be produced in large amounts are introduced into the mutant generated. It is conceivable that the promoters on the clusters to be introduced have to be replaced by promoters on the original cluster. The production of large amounts of a natural substance will be successful when the biosynthesis starting compounds of the original natural substance and of the novel natural substance are identical.

1. A recombination system for excision, integration, inversion or translocation of DNA fragments from/into the genome (target DNA) of actinomycetes, characterized in that it has at least one nucleic acid molecule which can be replicated in host cells and contains at least one nucleic acid sequence, selected from the group consisting of:
i) DNA sequences having at least 80% identity to the nucleotide sequence SEQ ID No:4 over the total length and coding for a Dre recombinase,
ii) DNA sequences having at least 80% identity to the nucleotide sequence SEQ ID No:5 over the total length and coding for a Cre recombinase,
and at least one identification sequence selected from the group consisting of: lox P and rox identification sequences in the target DNA.

2. The recombination system according to claim 1, characterized in that at least 70% of the nucleotide triplets from the groups i) and ii) represent codons that are preferred by actinomycetes.

3. The recombination system according to claim 1, characterized in that it can be used in an Escherichia coli or actinomycetes strain.

4. The recombination system according to claim 1, characterized in that it can be replicated in a Streptomycetes strain.

5. The recombination system according to claim 4, characterized in that the host cell streptomycetes strain is selected from the group consisting of: Streptomyces lividans, Streptomyces coelicolor, Streptomyces albus, Streptomyces cinnamonensis, Actinoplanes sp. SE50/110, Streptomyces galilaeus, Streptomyces nodosus, Actinosynnema pretiosum subsp. Auranticum, Streptomyces virido-chromogenes Tü57, Streptomyces echinatus, Streptomyces avermitilis MA-4680, Micromonospora echinospora, Streptomyces sp. FR-008, Streptomyces chartreusis, Streptomyces bikiniensis, Streptomyces roseochromogenes subsp. oscitans DS 12.976, Streptomyces griseus subsp. griseus, Streptomyces neyagawaensis, Streptomyces olindensis, Streptomyces rishiriensis DSM 40489, Streptomyces venezuelae, Streptomyces peucetius, Streptomyces olivaceus Tü2353, Saccharopolyspora erythraea, Micromonospora carbonacea var. Africana ATCC39149, Streptomyces griseoflavus Gö3592, Streptomyces ambofaciens, Streptomyces violaceoruber Tü22, Amycolatopsis orientalis, Amycolatopsis orientalis, Amycolatopsis mediterranei (balhimycina) DSM5908, Micromonospora echinospora ATCC 15835, Streptomyces griseoruber, Streptomyces aureofaciaens Tü11, Streptomyces antibioticus Tü99, Streptomyces antibioticus Tü1718, Streptomyces sp. Tü2187, Streptomyces sp. Tü1156, Streptomyces sp. FU107, Streptomyces sp. FU36, Streptomyces venezuelae ISP5230, Streptomyces kanamyceticus, Streptomyces cyanogenus S136, Streptomyces aureofaciens Tü117, Streptomyces rochei, Streptomyces globisporus 1912, Streptomyces sp. AM-7161, Micromonospora megalomicea, Streptomyces argillaceus, Streptomyces carzinostaticus ATCC 15944, Streptomyces fradiae NCIB 8233, Streptomyces spheroides,
NCIMB 11891, Streptomyces noursei ATCC11455, Streptomyces antibioticus, Actinoplanes teichomyceticus, Streptomyces natalensis, Streptomyces sp. Tü 6071, Streptomyces diastato-chromogenes Tü6028, Lechevalieria aerocolonigenes ATCC39243, Saccharothrix espanaensis, Micromonospora sp. Tü 6368, Streptomyces antibioticus Tü6040, Streptomyces nogalater, Sorangium cellulosum So ce12, Saccharopolyspora, Spinosa, Streptomyces sp. TP-A0274, Streptomyces steffisburgensis NRRL 3193, Streptomyces griseus, Streptomyces fradice, Streptomyces fradiae Tü2717, and Streptomyces halstedii HC-34.

6. The recombination system according to claim 1, characterized in that it represents a plasmid.

7. The recombination system according to claim 1, characterized in that the plasmid has an ErmE or tipA promoter.

8. The recombination system according to claim 1, characterized in that the plasmid is selected from the group consisting of: pUWL-T-Dre, pUWL-A-Dre, pUWL-H-Dre, pNL1-Der, pML1-Dre and pAL1-Dre, pUWL-T-Cre, pUWL-A-Cre, pUWL-H-Cre, pNL1-Cre, pML1-Cre and pAL1-Cre.

9. The recombination system according to claim 9, characterized in that the target DNA contains at least two identification sequences selected from the group consisting of loxP and rox identification sequences.

10. A method for producing modified actinomycetes, comprising the following steps:
a) inserting at least one identification sequence into the genome (target DNA) of the actinomycetes, the identification sequences being selected from the group consisting of loxP and rox identification sequences,
b) introducing at least one plasmid into the actinomycetes, the plasmid comprising a replicatable nucleic acid molecule coding for a codon-optimized recombinase, and
c) replicating the plasmid from b) and expressing the codon-optimized recombinase that is specific for the identification sequence(s) of the target DNA, excises, translocates or inverts a DNA fragment of the target DNA or inserts a DNA fragment into the target DNA.

11. The method according to claim 10, characterized in that the insertion of the identification sequences into the target DNA is effected with the use of a further plasmid, the plasmid containing the identification sequence and an attP identification sequence.

12. The method according to claim 10, characterized in that at least two identification sequences selected from the group consisting of: IoxP and rox identification sequences are introduced into the target DNA.

13. The method according to claim 10, characterized in that at least two different nucleic acid molecules according to claim 1 are used.

14. (canceled)

15. Modified microorganisms produced by the method according to claim 10.


SEQUENCE LISTING (12 sequences)

SEQ ID NO: 1 - DNA, 1029 bp, Artificial sequence
  retrofitting vector pRetroES
atgagtgaattaattatctctggcagttccggtggttttctgcgcaacattggcaaagagtaccaggaagccgcagaaaactttatgcggttcatgaatgatcagggtgcttatgcgccgaatactctgcgcgacctccggttggtgtttcattcctgggcgcgctggtgtcacgctcgccagcttgcctggtttccgatctcaccagaaatggcccgcgagtattttcttcagttgcatgatgccgatctggcttcgaccaccattgataagcactacgccatgcttaacatgctgctttctcattgtggccttccgccactttcggatgataaaagtgtttctctggctatgcggcgtatccggcgcgaagcggcaacggaaaaaggcgaacgaacaggccaggctataccgctgcgatgggacgacctgaaactgctcgacgtcctgttgtccaggtcagaacggctggtggacctgcgcaaccgggcttttctctttgttgcatacaatacgctgatgcgtatgtcggaaatctcgcgtattcgtgtaggagatctggaccaaacaggtgacactgtcacgctacatatttcacacacgaagacaataacgaccgccgccgggcttgataaggtgctttcccgtcgcactactgctgtgctgaatgactggctggatgtttccgggcttcgtgaacatcctgacgcggtgctgttcccgccgatacaccgtagcaataaggccaggattacgacaacgccccttactgcacctgcaatggagaaaatattcagcgacgcctgggtgttgctgaataaaagggatgccacgccaaacaaagggagataccggacgtggaccgggcatagtgctcgtgtcggggccgctatcgatatggctgaaaagcaggtgtctatggtggagattatgcaggaaggaacctggaagaaaccggaaacacttatgcggtatctgcgccgtggcggtgtgtctgtgggggctaatagccgcttgatggattcataa

SEQ ID NO: 2 - DNA, 1032 bp, Artificial sequence
  retrofitting vector pRetroES
atgtccaatttactgaccgtacaccaaaatttgcctgcattaccggtcgatgcaacgagtgatgaggttcgcaagaacctgatggacatgttcagggatcgccaggcgttttctgagcatacctggaaaatgcttctgtccgtttgccggtcgtgggcggcatggtgcaagttgaataaccggaaatggtttcccgcagaacctgaagatgttcgcgattatcttctatatcttcaggcgcgcggtctggcagtaaaaactatccagcaacatttgggccagctaaacatgcttcatcgtcggtccgggctgccacgaccaagtgacagcaatgctgtttcactggttatgcggcggatccgaaaagaaaacgttgatgccggtgaacgtgcaaaacaggctctagcgttcgaacgcactgatttcgaccaggttcgttcactcatggaaaatagcgatcgctgccaggatatacgtaatctggcatttctggggattgcttataacaccctgttacgtatagccgaaattgccaggatcagggttaaagatatctcacgtactgacggtgggagaatgttaatccatattggcagaacgaaaacgctggttagcaccgcaggtgtagagaaggcacttagcctgggggtaactaaactggtcgagcgatggatttccgtctctggtgtagctgatgatccgaataactacctgttttgccgggtcagaaaaaatggtgttgccgcgccatctgccaccagccagctatcaactcgcgccctggaagggatttttgaagcaactcatcgattgatttacggcgctaaggatgactctggtcagagatacctggcctggtctggacacagtgcccgtgtcggagccgcgcgagatatggcccgcgctggagtttcaataccggagatcatgcaagctggtggctggaccaatgtaaatattgtcatgaactatatccgtaacctggatagtgaaacaggggcaatggtgcgcctgctggaagatggcgattag

SEQ ID NO: 3 - DNA, 1272 bp, Saccharomyces cerevisiae
atgccacaatttgatatattatgtaaaacaccacctaaggtgcttgttcgtcagtttgtggaaaggtttgaaagaccttcaggtgagaaaatagcattatgtgctgctgaactaacctatttatgttggatgattacacataacggaacagcaatcaagagagccacattcatgagctataatactatcataagcaattcgctgagtttggatattgtcaacaagtcactgcagtttaaatacaagacgcaaaaagcaacaattctggaagcctcattaaagaaattgattcctgcttgggaatttacaattattccttactatggacaaaaacatcaatctgatatcactgatattgtaagtagtttgcaattacagttcgaatcatcggaagaagcagataagggaaatagccacagtaaaaaaatgcttaaagcacttctaagtgagggtgaaagcatctgggagatcactgagaaaatactaaattcgtttgagtatacttcgagatttacaaaaacaaaaactttataccaattcctcttcctagctactttcatcaattgtggaagattcagcgatattaagaacgttgatccgaaatcatttaaattagtccaaaataagtatctgggagtaataatccagtgtttagtgacagagacaaagacaagcgttagtaggcacatatacttctttagcgcaaggggtaggatcgatccacttgtatatttggatgaatttttgaggaattctgaaccagtcctaaaacgagtaaataggaccggcaattcttcaagcaacaagcaggaataccaattattaaaagataacttagtcagatcgtacaacaaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataa

SEQ ID NO: 4 - DNA, 1029 bp, Artificial sequence
  retrofitting vector pRetroES
atgtccgagctgatcatctccggctcctccggcggcttcctgcgcaacatcggcaaggagtaccaggaggccgccgagaacttcatgcgcttcatgaacgaccagggcgcctacgccccgaacaccctgcgcgacctgcgcctggtcttccactcctgggcccgctggtgccacgcccgccagctggcctggttcccgatctccccggagatggcccgcgagtacttcctgcagctgcacgacgccgacctggcctccaccaccatcgacaagcactacgccatgctgaacatgctgctgtcccactgcggcctgccgccgctgtccgacgacaagtccgtctccctggccatgcgccgcatccgccgcgaggccgccaccgagaagggcgagcgcaccggccaggccatcccgctgcgctgggacgacctgaagctgctggacgtcctgctgtcccgctccgagcgcctggtcgacctgcgcaaccgcgccttcctgttcgtcgcctacaacaccctgatgcgcatgtccgagatctcccgcatccgcgtcggcgacctggaccagaccggcgacaccgtcaccctgcacatctcccacaccaagaccatcaccaccgccgccggcctggacaaggtcctgtcccgccgcaccaccgccgtcctgaacgactggctggacgtctccggcctgcgcgagcacccggacgccgtcctgttcccgccgatccaccgctccaacaaggcccgcatcaccaccaccccgctgaccgccccggccatggagaagatcttctccgacgcctgggtcctgctgaacaagcgcgacgccaccccgaacaagggccgctaccgcacctggaccggccactccgcccgcgtcggcgccgccatcgacatggccgagaagcaggtctccatggtcgagatcatgcaggagggcacctggaagaagccggagaccctgatgcgctacctgcgccgcggcggcgtctccgtcggcgccaactcccgcctgatggactcctga

SEQ ID NO: 5 - DNA, 1032 bp, Artificial sequence
  retrofitting vector pRetroES
atgtccaacctgctgaccgtccaccagaacctgccggccctgcccgtggacgccaccagcgacgaggtccggaagaacctgatggacatgttccgggaccggcaggccttctccgagcacacctggaagatgctgctgtccgtgtgccgctcgtgggccgcgtggtgcaagctgaacaaccggaagtggttcccggcggagccggaggacgtgcgcgactacctcctgtacctccaggcccggggcctggcggtcaagaccatccagcagcacctgggccagctgaacatgctgcaccgccggtcgggtctgccgcgccccagcgacagcaacgcggtcagcctggtcatgcggcgcatccggaaggaaaacgtcgacgccggggagcgcgccaagcaggcgctggcgttcgagcgcaccgacttcgaccaggtccgcagcctcatggagaactcggaccggtgccaggacatccggaacctggcgttcctgggtatcgcgtacaacaccctgctccgcatcgccgagatcgcgcgcatccgcgtgaaggacatcagccggaccgacggtggccgcatgctgatccacatcgggcggacgaagaccctcgtctccaccgccggcgtcgagaaggcgctctcgctcggcgtgaccaagctcgtggagcgctggatctccgtctccggcgtcgccgacgacccgaacaactacctgttctgccgcgtgcggaagaacggcgtggccgccccctccgcgacctcccagctctccacgcgcgccctggagggcatcttcgaggccacgcaccgcctcatctacggggccaaggacgactccggccagcggtacctggcgtggtcgggccactccgcgcgcgtcggtgccgcgcgggacatggcccgcgcgggggtgtcgatcccggagatcatgcaggccggcgggtggaccaacgtcaacatcgtcatgaactacatccgcaacctggactcggagacgggcgcgatggtgcgcctgctggaagacggcgactga

SEQ ID NO: 6 - DNA, 1272 bp, Artificial sequence
  retrofitting vector pRetroES
atgccgcagttcgacatcctctgcaagaccccgcccaaggtgctcgtgcggcagttcgtcgagcgcttcgagcgcccgagcggcgagaagatcgccctgtgcgccgccgagctgacctacctctgctggatgatcacccacaacggcacggcgatcaagcgggccaccttcatgtcctacaacaccatcatctccaactcgctcagcctcgacatcgtgaacaagtccctccagttcaagtacaagacccagaaggcgaccatcctggaagcgtccctgaagaagctgatcccggcgtgggagttcaccatcatcccctactacggccagaagcaccagtccgacatcaccgacatcgtctcctcgctccagctccagttcgagtcgtccgaggaggccgacaagggcaactcccactccaagaagatgctgaaggcgctgctgagcgagggcgagtccatctgggagatcacggagaagatcctgaactccttcgagtacacctcccggttcaccaagacgaagaccctctaccagttcctgttcctggcgaccttcatcaactgcggccggttcagcgacatcaagaacgtcgacccgaagtccttcaagctggtccagaacaagtacctgggcgtcatcatccagtgcctggtcaccgagacgaagacctccgtgtcgcggcacatctacttcttctccgcgcggggccgcatcgacccgctggtctacctcgacgagttcctgcgcaactcggagccggtcctcaagcgcgtcaaccggaccggcaacagcagctccaacaagcaggagtaccagctgctcaaggacaacctggtccggtcgtacaacaaggccctgaagaagaacgccccgtactccatcttcgcgatcaagaacggcccgaagagccacatcggtcgccacctgatgacctcgttcctgtccatgaaggggctgaccgagctcaccaacgtcgtcggtaactggagcgacaagcgcgcctccgccgtggcgcggaccacgtacacccaccagatcacggccatcccggaccactacttcgccctggtgtcgcgctactacgcctacgacccgatctccaaggaaatgatcgccctgaaggacgagacgaacccgatcgaggagtggcagcacatcgagcagctgaagggttccgccgagggctcgatccggtacccggcctggaacggtatcatctcgcaggaggtgctggactacctgagcagctacatcaaccggcggatctga

SEQ ID NO: 7 - DNA, 34 bp, Artificial sequence
  retrofitting vector pRetroES
ataacttcgtataatgtatgctatacgaagttat

SEQ ID NO: 8 - DNA, 32 bp, Artificial sequence
  retrofitting vector pRetroES
taactttaaataatgccaattatttaaagtta

SEQ ID NO: 9 - DNA, 34 bp, Artificial sequence
  retrofitting vector pRetroES
gaagttcctattctctagaaagtataggaacttc

SEQ ID NO: 10 - protein, 342 aa, Artificial sequence
  retrofitting vector pRetroES
MSELIISGSSGGFLRNIGKEYQEAAENFMRFMNDQGAYAPNTLRDLRLVFHSWARWCHARQLAWFPISPEMAREYFLQLHDADLASTTIDKHYAMLNMLLSHCGLPPLSDDKSVSLAMRRIRREAATEKGERTGQAIPLRWDDLKLLDVLLSRSERLVDLRNRAFLFVAYNTLMRMSEISRIRVGDLDQTGDTVTLHISHTKTITTAAGLDKVLSRRTTAVLNDWLDVSGLREHPDAVLFPPIHRSNKARITTTPLTAPAMEKIFSDAWVLLNKRDATPNKGRYRTWTGHSARVGAAIDMAEKQVSMVEIMQEGTWKKPETLMRYLRRGGVSVGANSRLMDS

SEQ ID NO: 11 - protein, 343 aa, Artificial sequence
  retrofitting vector pRetroES
MSNLLTVHQNLPALPVDATSDEVRKNLMDMFRDRQAFSEHTWKMLLSVCRSWAAWCKLNNRKWFPAEPEDVRDYLLYLQARGLAVKTIQQHLGQLNMLHRRSGLPRPSDSNAVSLVMRRIRKENVDAGERAKQALAFERTDFDQVRSLMENSDRCQDIRNLAFLGIAYNTLLRIAEIARIRVKDISRTDGGRMLIHIGRTKTLVSTAGVEKALSLGVTKLVERWISVSGVADDPNNYLFCRVRKNGVAAPSATSQLSTRALEGIFEATHRLIYGAKDDSGQRYLAWSGHSARVGAARDMARAGVSIPEIMQAGGWTNVNIVMNYIRNLDSETGAMVRLLEDGD

SEQ ID NO: 12 - protein, 423 aa, Saccharomyces cerevisiae
MPQFDILCKTPPKVLVRQFVERFERPSGEKIALCAAELTYLCWMITHNGTAIKRATFMSYNTIISNSLSLDIVNKSLQFKYKTQKATILEASLKKLIPAWEFTIIPYYGQKHQSDITDIVSSLQLQFESSEEADKGNSHSKKMLKALLSEGESIWEITEKILNSFEYTSRFTKTKTLYQFLFLATFINCGRFSDIKNVDPKSFKLVQNKYLGVIIQCLVTETKTSVSRHIYFFSARGRIDPLVYLDEFLRNSEPVLKRVNRTGNSSSNKQEYQLLKDNLVRSYNKALKKNAPYSIFAIKNGPKSHIGRHLMTSFLSMKGLTELTNVVGNWSDKRASAVARTTYTHQITAIPDHYFALVSRYYAYDPISKEMIALKDETNPIEEWQHIEQLKGSAEGSIRYPAWNGIISQEVLDYLSSYINRRI